(19) United States
(12) Patent Application Publication
(10) Pub. No.: US 2010/0196287 A1
(43) Pub. Date: Aug. 5, 2010

(54) Compositions Comprising Perhydrolases and Alkylene Glycol Diacetates

(76) Inventors: Timothy O'Connell, Dusseldorf (DE); Karl-Heinz Maurer, Erkrath (DE); Thomas Weber, Dormagen (DE); Inken Prüser, Dusseldorf (DE)

Correspondence Address: Henkel Corporation, 10 Finderne Avenue, Bridgewater, NJ, 08807, US

(21) Appl. No.: 12/697,360

(22) Filed: Feb. 1, 2010

Related U.S. Application Data
(63) Continuation of application No. PCT/EP2008/058120, filed on Jun. 26, 2008.

(30) Foreign Application Priority Data
Jul. 31, 2007 (DE) 10 2007 036 392.5

(51) Int. Cl. C11D 3/386; A61K 38/46

(52) U.S. Cl. 424/50; 510/116; 510/392; 510/393; 510/321; 510/320; 510/226; 510/235; 424/94.6

(57) ABSTRACT

The present invention relates to compositions, in particular washing and cleaning agents as well as cosmetic and pharmaceutical preparations, comprising perhydrolases and alkylene glycol diacetates.

### CROSS REFERENCE TO RELATED APPLICATIONS

The present application is a continuation of International Application No. PCT/EP2008/058120, filed 26 Jun. 2008, which claims priority to German Patent Application No. 10 2007 036 392.5, filed 31 Jul. 2007.

The present invention relates to compositions, in particular, washing and cleaning agents as well as cosmetic and pharmaceutical preparations, comprising perhydrolases and alkylene glycol diacetates.

Perhydrolases, which in the past were also called metal-free haloperoxidases, generally comprise the catalytic triad Ser-His-Asp in the reaction center and catalyze the reversible formation of peroxy acids starting from carboxylic acids and hydrogen peroxide.

A disadvantage of most known highly active perhydrolase substrates is that they exhibit properties that make them unusable in washing agents. These properties include insufficient storage stability, insufficient solubility and/or stability in water (particularly in the presence of hydrogen peroxide), low activity, poor producibility or availability, easy explosiveness or flammability, unwanted toxic, carcinogenic, teratogenic or ecological characteristics, as well as unwanted colors or odor.

Accordingly, the present invention identifies new substrates that possess high activity in combination with perhydrolases and, at the same time, are suitable for use in washing agents.

It has now been surprisingly found that short chain alkylene glycol diacetates achieve the object of the invention, even better than other similarly simply constructed substrates.

### BRIEF DESCRIPTION OF THE DRAWINGS

FIG. 1 is a graph illustrating washing results from use of 50 mM of ethylene glycol diacetate and propylene glycol diacetate in comparison with methyl acetate on removing blueberry and tea stains from fabric.

Accordingly, a first subject matter of the present invention is compositions comprising at least one perhydrolase and at least one C₂₋₆ alkylene glycol diacetate.

In this regard, the perhydrolase is preferably an enzyme chosen from lipases, esterases and proteases, wherein the enzyme is capable of forming peroxy acids starting from carboxylic acids and hydrogen peroxide. In a preferred embodiment, the reaction center of the perhydrolase comprises the catalytic triad Ser-His-Asp.

In regard to inventively suitable perhydrolases, reference is particularly made to applications DE 10260903, DE 102004029475, WO 98/45398, WO 04/58961, WO 05/56782, WO 05/124012 and US 2007105740.

According to the invention, the perhydrolase is preferably added as an enzyme concentrate or as a solid perhydrolase preparation in quantities of 0.1 to 5.0 wt. %, preferably 0.2 to 3.0 wt. %, each based on total weight of the composition.

In particular, a compound chosen from ethylene glycol diacetate, propylene glycol diacetate, butylene glycol diacetate, pentylene glycol diacetate and hexylene glycol diacetate can be employed as the C₂₋₆ alkylene glycol diacetate. Ethylene glycol diacetate or propylene glycol diacetate are preferably employed.

C₂₋₆ alkylene glycol diacetate is preferably found in compositions according to the invention in an amount of 0.01 to 10 wt. %, preferably in an amount of 0.01 to 5 wt. %, more preferably in an amount of 0.05 to 4 wt. %, and most preferably in an amount of 0.1 to 3 wt. %, each based on total weight of the composition.

In addition to the C₂₋₆ alkylene glycol diacetate, compositions according to the invention can also further include carboxylic acid esters, carboxylic acids and/or their salts and/or derivatives thereof as the substrate for the perhydrolase. Generally, one may cite exemplary compounds of the Formula R¹—(R²)ₘ—OR³, wherein
R¹ is R⁴C(O)— or R⁴C(NR⁵)—,
R³ is hydrogen or optionally substituted, particularly by optionally substituted amino group substituted alkyl, alkenyl, alkynyl, aryl, alkylaryl, heteroaryl or alkylheteroaryl or for a group —X—O—(R^{2b})ₙ—R^{1b},
R⁴ is hydrogen or optionally substituted alkyl, alkenyl, alkynyl, aryl, alkylaryl, heteroaryl or alkylheteroaryl or for a group —Y—R^{1a}—(R^{2a})ₒ—OR^{3a},
X and Y are optionally substituted alkyl, alkenyl, alkynyl, aryl, alkylaryl, heteroaryl or alkylheteroaryl,
R², R^{2a} and R^{2b} are an alkoxy group,
R^{1a} is —C(O)— or —C(NR⁵)—,
R^{1b} is R⁶C(O)— or R⁶C(NR⁵)—,
R^{1a} and R⁶ are hydrogen or optionally substituted alkyl, alkenyl, alkynyl, aryl, alkylaryl, heteroaryl or alkylheteroaryl,
R⁵ is hydrogen or alkyl,
m, n and o, independently, are a value from 0 to 12.

In particular, acetic acid, propionic acid, butyric acid, valeric acid, hexanoic acid, heptanoic acid, octanoic acid, decanoic acid, maleic acid, oxalic acid, benzoic acid, citric acid, lactic acid, fruit acids and phthalic acid may be cited as examples of carboxylic acids, optionally in the form of their esters and/or salts, that can be inventively employed as the additional perhydrolase substrate.

Hydrogen peroxide can be added to compositions according to the invention as is, or additionally or alternatively a bleaching agent that releases hydrogen peroxide can be employed. Among those compounds which serve as bleaching agents and liberate H₂O₂ in water, sodium percarbonate, sodium perborate tetrahydrate and sodium perborate monohydrate are of particular importance. Examples of further bleaching agents that may be used include peroxypyrophosphates, citrate perhydrates and H₂O₂ liberating peracidic salts or peracids, such as persulfates or persulfuric acid. Urea peroxyhydrate percarbamide, described by the formula H₂N—CO—NH₂.H₂O₂, is also suitable. When agents are used to clean hard surfaces, for example, in automatic dishwashers, they can also include organic bleaching agents, although they are typically used for washing fabrics. Typical organic bleaching agents include diacyl peroxides such as dibenzoyl peroxide. Further typical organic bleaching agents include peroxy acids such as alkylperoxy acids and arylperoxy acids. Preferred representatives that can be added include (a) peroxybenzoic acid and ring substituted derivatives thereof such as alkyl peroxybenzoic acids, as well as peroxy-a-naphthoic acid and magnesium monoperphthalate, (b) aliphatic or substituted aliphatic peroxy acids such as peroxylauric acid, peroxystearic acid, ε-phthalimidoperoxycaproic acid [phthaloiminoperoxyhexanoic acid (PAP)], o-carboxybenzamido peroxycaproic acid, N-nonenylamido peradipic acid and N-nonenylamido persuccinates, and (c) aliphatic and araliphatic peroxydicarboxylic acids such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-dioic acid, and N,N-terephthaloyl-di(6-aminopercaproic acid).

Chlorine- or bromine-releasing substances can also be used as bleaching agents. Suitable chlorine- or bromine-releasing materials include heterocyclic N-bromamides and N-chloramides, (e.g., trichloroisocyanuric acid, tribromoisocyanuric acid, dibromoisocyanuric acid and/or dichloroisocyanuric acid (DICA) and/or salts thereof with cations such as potassium and sodium). Hydantoin compounds such as 1,3-dichloro-5,5-dimethyl hydantoin are also suitable.

According to the invention, compositions such as washing or cleaning agents, particularly automatic dishwasher detergents, are preferred that comprise up to 45 wt. %, particularly 1 to 35 wt. %, preferably 2.5 to 30 wt. %, particularly preferably 3.5 to 20 wt. % and especially 5 to 15 wt. % bleaching agent, preferably sodium percarbonate, based on total weight of the composition.

Active oxygen content of compositions according to the invention, particularly washing or cleaning agents, above all automatic dishwasher detergents, preferably ranges between 0.4 and 10 wt. %, particularly preferably between 0.5 and 8 wt. % and particularly between 0.6 and 5 wt. %, based on total weight of the composition. Particularly preferred agents possess an active oxygen content above 0.3 wt. %, preferably above 0.7 wt. %, particularly preferably above 0.8 wt. % and particularly above 1.0 wt. %, based on total weight of the composition.

Alternatively, in order to supply hydrogen peroxide, oxidoreductase enzymes can also be employed to produce hydrogen peroxide in situ. The catalyzed reaction is the transfer of electrons from the organic substrate, for the glucose oxidase, for example, from the glucose, to the oxygen as the electron acceptor with the formation of the desired hydrogen peroxide. Accordingly, in a preferred embodiment, compositions according to the invention comprise at least one oxidoreductase that produces hydrogen peroxide.

The hydrogen peroxide-producing oxidoreductases here concern an oxidoreductase that produces hydrogen peroxide, using oxygen as an electron acceptor. In this regard, particularly preferred oxidoreductases include those of the EC classes E.C. 1.1.3 (CH—OH as the electron donor), E.C. 1.2.3 (aldehyde or oxo groups as the electron donor), E.C. 1.4.3 (CH—NH₂ as the donor), E.C. 1.7.3 (N-containing groups as the donor) and E.C. 1.8.3 (S-containing groups as the donor) come into consideration, wherein enzymes of the EC class EC 1.1.3.

Preferred enzymes are chosen in particular from malate oxidase (EC 1.1.3.3), glucose oxidase (EC 1.1.3.4), hexose oxidase (EC 1.1.3.5), cholesterin oxidase (EC 1.1.3.6), galactose oxidase (EC 1.1.3.9), pyranose oxidase (EC 1.1.3.10), alcohol oxidase (EC 1.1.3.13), choline oxidase (EC 1.1.3.17, see in particular WO 04/58955), oxidases for long chain alcohols (EC 1.1.3.20), glycerin-3-phosphate oxidase (EC 1.1.3.21), cellobiose oxidase (EC 1.1.3.25), nucleoside oxidase (EC 1.1.3.39), D-mannitol oxidase (EC 1.1.3.40), xylitol oxidase (EC 1.1.3.41), aldehyde oxidase (EC 1.2.3.1), pyruvate oxidase (EC 1.2.3.3), oxalate oxidase (EC 1.2.3.4), glyoxylate oxidase (EC 1.2.3.5), indole-3-acetaldehyd oxidase (EC 1.2.3.7), pyridoxal oxidase (EC 1.2.3.8), arylaldehyde oxidase (EC 1.2.3.9), retinal oxidase (EC 1.2.3.11), L-amino acid oxidase (EC 1.4.3.2), amine oxidase (EC 1.4.3.4, EC 1.4.3.6), L-glutamate oxidase (EC 1.4.3.11), L-lysine oxidase (EC 1.4.3.14), L-aspartate oxidase (EC 1.4.3.16), tryptophan-α,β oxidase (EC 1.4.3.17), glycine oxidase EC 1.4.3.19), urea oxidase (EC 1.7.3.3), thiol oxidase (EC 1.8.3.2) and glutathione oxidase (EC 1.8.3.3).

In a preferred embodiment, the hydrogen peroxide-producing oxidoreductase is one in which a sugar is used as the electron donor. The hydrogen peroxide-producing and sugar-oxidizing oxidoreductase is preferably chosen from glucose oxidase (EC 1.1.3.4), hexose oxidase (EC 1.1.3.5), galactose oxidase (EC 1.1.3.9) and pyranose oxidase (EC 1.1.3.10). According to the invention, glucose oxidase (EC 1.1.3.4) is particularly preferred. Advantageously, additional, preferably organic, particularly preferably aromatic compounds are added that interact with the enzymes to enhance the activity of the oxidoreductases (Enhancer) or to facilitate electron flow (Mediators) between the oxidizing enzymes and the stains over strongly different redox potentials.

Hydrogen peroxide-producing oxidoreductases are preferably incorporated into washing and cleaning agents according to the invention in an amount such that the total composition exhibits an enzyme activity in relation to the oxidoreductases of 30 U/g to 20 000 U/g, in particular from 60 U/g to 15 000 U/g. The unit 1 U here corresponds to the activity of a quantity of enzyme that converts 1 μmol of its substrate at pH 7 and 25° C. in one minute.

In addition to hydrogen peroxide-producing oxidoreductases, the inventive compositions can also comprise additional oxidoreductases, in particular oxidases, oxygenases, laccases (phenoloxidase, polyphenoloxidases) and/or dioxygenases. Denilite® 1 and 2, available from the Novozymes Company, are examples of suitable commercial products for laccases. In a preferred embodiment, the additional oxidoreductase is chosen from enzymes that use peroxides as the electron accepter (EC-Classes 1.11 or 1.11.1), in particular, from catalases (EC 1.11.1.6), peroxidases (EC 1.11.1.7), glutathione peroxidases (EC 1.11.1.9), chloride peroxidases (EC 1.11.1.10), manganese peroxidases (EC 1.11.1.13) and/or lignin peroxidases (EC 1.11.1.14), which can also be generally classified under the term peroxidases.

Compositions according to the invention can additionally comprise bleach auxiliaries selected from bleach activators and bleach catalysts for boosting the bleaching power. Useful bleach activators include compounds which, under perhydrolysis conditions, yield aliphatic peroxycarboxylic acids having preferably 1 to 10 carbon atoms, in particular 2 to 4 carbon atoms, and/or optionally substituted perbenzoic acid. Substances which carry O-acyl and/or N-acyl groups of said number of carbon atoms and/or optionally substituted benzoyl groups, are suitable. Preference is given to polyacylated alkylenediamines, in particular, tetraacetyl ethylenediamine (TAED), acylated triazine derivatives, in particular, 1,5-diacetyl-2,4-dioxohexahydro-1,3,5-triazine (DADHT), acylated glycolurils, in particular, tetraacetyl glycoluril (TAGU), N-acylimides, in particular N-nonanoyl succinimide (NOSI), acylated phenol sulfonates, in particular n-nonanoyl or isononanoyloxybenzene sulfonate (n- or iso-NOBS), acylated hydroxycarboxylic acids such as triethyl-O-acetyl citrate (TEOC), carboxylic acid anhydrides, in particular, phthalic anhydride, isatoic anhydride and/or succinic anhydride, carboxylic acid amides such as N-methyldiacetamide, glycolide, acylated polyhydric alcohols, in particular, triacetin, ethylene glycol diacetate, isoprenyl acetate and 2,5-diacetoxy-2,5-dihydrofuran and enol esters known from the German Patent Applications DE 196 16 693 and DE 196 16 767 and acetylated sorbitol and mannitol or their mixtures (SORMAN) described in European Patent Application EP 0 525 239, acylated sugar derivatives, in particular, pentaacetyl glucose (PAG), pentaacetyl fructose, tetraacetyl xylose and octaacetyl lactose as well as acetylated, optionally N-alkylated glucamine and gluconolactone, triazole or triazole derivatives and/or particulate caprolactams and/or caprolactam derivatives, preferably, N-acylated lactams, (e.g., N-benzoylcaprolactam and N-acetyl caprolactam). Hydrophilically substituted acyl acetals and acyl lactams are also preferably used. Combinations of conventional bleach activators may also be used. Nitrile derivatives such as cyanopyridines, nitrilequats (e.g., N-alkylammonium acetonitrile), and/or cyanamide derivatives can also be used. Preferred bleach activators include sodium 4-(octanoyloxy)benzene sulfonate, n-nonanoyl— or isononanoyloxybenzene sulfonate (n- or iso-NOBS), undecenoyloxybenzene sulfonate (UDOBS), sodium dodecanoyloxybenzene sulfonate (DOBS), decanoyloxybenzoic acid (DOBA, OBC 10) and/or dodecanoyloxybenzene sulfonate (OBS 12), and N-methylmorpholinum acetonitrile (MMA).

In the context of the present application, further preferred employable bleach activators include cationic nitriles, particularly cationic nitriles of the Formula

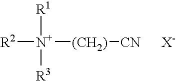

wherein R¹ is —H, —CH₃, a C₂₋₂₄ alkyl or alkenyl group, a substituted C₂₋₂₄ alkyl or alkenyl group having at least one substituent from the group of —Cl, —Br, —OH, —NH₂, —CN, an alkyl or alkenylaryl group having a C₁₋₂₄ alkyl group, or for an alkyl or alkenylaryl group substituted with a C₁₋₂₄ alkyl group and at least one further substituent on the aromatic ring; R² and R³, independently, are —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, or —(CH₂CH₂—O)ₙH with n=1, 2, 3, 4, 5 or 6; and X is an anion.

A cationic nitrile of the following Formula is particularly preferred —

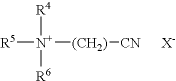

wherein R⁴, R⁵ and R⁶, independently, are —CH₃, —CH₂—CH₃, —CH₂—CH₂—CH₃ or —CH(CH₃)—CH₃, wherein R⁴ can also be —H; and X is an anion; wherein preferably R⁵═R⁶═—CH₃, and in particular, R⁴═R⁵═R⁶═—CH₃; and compounds of the formulae (CH₃)₃N⁽⁺⁾CH₂—CN X⁻, (CH₃CH₂)₃N⁽⁺⁾CH₂—CN X⁻, (CH₃CH₂CH₂)₃N⁽⁺⁾CH₂—CN X⁻, (CH₃CH(CH₃))₃N⁽⁺⁾CH₂—CN X⁻, or (HO—CH₂—CH₂)₃N⁽⁺⁾CH₂—CN X⁻ are particularly preferred; wherein from the group of these substances the cationic nitrile of the formula (CH₃)₃N⁽⁺⁾CH₂—CN X⁻, in which X⁻ is an anion chosen from chloride, bromide, iodide, hydrogen sulfate, methosulfate, p-toluene sulfonate (tosylate) or xylene sulfonate, is particularly preferred.

In a particularly preferred embodiment, the bleach activator is TAED.

Bleach activators are preferably present in compositions according to the invention, in particular, in washing and cleaning agents, in an amount of 0.01 to 20 wt. %, more preferably in an amount of 0.1 to 15 wt. %, in particular, in an amount of 1 to 10 wt. %, and above all, in an amount of 2 to 5 wt. %, based on weight of the total composition.

In addition to or instead of the conventional bleach activators mentioned above, bleach catalysts may be incorporated. In general, these substances include any bleach-boosting transition metal salt or any transition metal complex. In particular, manganese, iron, cobalt, ruthenium, molybdenum, titanium or copper in various oxidation states are suitable transition metals. In particular, guanidines, aminophenols, amine oxides, salenes, saldimines, lactams, monocyclic and cross-bridged polycyclic polyazaalkanes, terpyridines, dendrimers, tetraamido ligands, bis- and tetrakis(pyridylmethyl)alkylamines, secondary amines and polyoxometallates, as described in the literature, are possible complexing ligands.

Bleach-boosting transition metal complexes, particularly those containing the central atoms Mn, Fe, Co, Cu, Mo, V, Ti and/or Ru, preferably manganese and/or cobalt salts and/or complexes, particularly preferably cobalt(ammine) complexes, cobalt(acetate) complexes, cobalt(carbonyl) complexes, chlorides of cobalt or manganese and manganese sulfate, are used in typical quantities, preferably in a quantity of up to 5 wt. %, especially in a quantity of 0.0025 wt. % to 1 wt. %, and particularly preferably in a quantity of 0.01 wt. % to 0.25 wt. %, each based on total weight of the bleach activator-containing agent. However, in special cases, more bleach activator may also be employed.

Manganese complexes in the valence state II, III, IV or V, which preferably comprise one or more macrocyclic ligands with donor functions N, NR, PR, O and/or S, are preferred. Ligands having nitrogen donor functions are preferably employed. In this regard, it is particularly preferred to incorporate bleach catalysts into compositions according to the invention comprising 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN) and/or 2-methyl-1,4,7-triazacyclononane (Me/TACN) as the macromolecular ligands. Suitable manganese complexes are for example [Mn^{III}₂(μ-O)₁(μ-OAc)₂(TACN)₂](ClO₄)₂, [Mn^{III}Mn^{IV}(μ-O)₂(μOAc)₁(TACN)₂](BPh₄)₂, [Mn^{IV}₄(μ-O)₆(TACN)₄](ClO₄)₄, [Mn^{III}₂(μ-O)₁(μ-OAc)₂(Me-TACN)₂](ClO₄)₂, [Mn^{III}Mn^{IV}(μ-O)₁(μ-OAc)₂(Me-TACN)₂](ClO₄)₃, [Mn^{IV}₂(μ-O)₃Me-TACN)₂](PF₆)₂ and [Mn^{IV}₂(μ-O)₃(Me/Me-TACN)₂](PF₆)₂(OAc═OC(O)CH₃).

Bleach catalysts can be added in typical amounts, preferably in an amount of up to 5 wt. %, particularly from 0.0025 wt. % to 1 wt. % and particularly preferably from 0.01 to 0.25 wt. %, each based on the total weight of the composition, particularly a washing or cleaning agent. However, in special cases more bleach activator may also be employed.

In particular, compositions according to the invention include washing or cleaning agents, as well as from cosmetic and pharmaceutical compositions.

### Washing and Cleaning Agents

In the broadest sense of the invention, washing and cleaning agents are understood to mean surfactant-containing preparations in solid form (particles, powder etc.), semi-solid form (pastes, etc.), liquid form (solutions, emulsions, suspensions, gels etc.) and gaseous-like form (aerosols etc.). These agents can comprise any type of surfactant, usually in addition to further components typical for each of the end uses. Preferably, agents according to the invention include surfactants chosen from non-ionic, anionic, cationic and amphoteric surfactants. According to the invention, non-ionic, anionic and/or amphoteric surfactants are more preferably employed as the surfactants. Examples of such surfactant-containing preparations include surfactant-containing washing agent preparations such as machine fabric-washing agents, hand washing agents or rinse agents, surfactant-containing cleaning agents for hard surfaces such as household cleaners, hand dishwashing agents or automatic dishwasher agents, surfactant-containing disinfectants and surfactant-containing freshening preparations. Each can be in solid or liquid form; however, they can also be in a form that includes solid and liquid components or partial amounts of the components alongside one another. Washing or cleaning agents according to the invention can include additional washing or cleaning active ingredients, described in greater detail below.

Generally, inventive washing or cleaning agents include one or more builders such as zeolites, silicates, carbonates, organic cobuilders and - where there are no environmental restrictions against their use - also phosphates. The last are particularly preferred builders employed in cleaning agents for automatic dishwashers.

Washing and cleaning agents according to the invention can optionally include other typical ingredients such as sequestering agents, electrolytes and further auxiliaries, such as polymers, additional enzymes, solvents, thickeners, optical brighteners, graying inhibitors, glass corrosion inhibitors, corrosion inhibitors, foam inhibitors, disintegration auxiliaries, abrasives, dyes and/or fragrances, as well as antimicrobial agents, UV absorbers, blowing agents and/or enzyme stabilizers.

Concerning suitable builders, surfactants, polymers, polymers, additional enzymes, enzyme stabilizers, solvents, thickeners, optical brighteners, graying inhibitors, corrosion inhibitors, soil release agents, color transfer inhibitors, foam inhibitors, abrasives, disintegration auxiliaries, acidifiers, dyes and fragrances, antimicrobials, UV-absorbers and blowing agents together with their preferred addition quantities, reference is made to International Patent Publication No. WO 2009/015951.

Processes for cleaning fabrics or hard surfaces comprise another aspect of the invention, wherein at least one combination of at least one perhydrolase and at least one C₂₋₆ alkylene glycol diacetate is used in at least one of the process steps. This pertains to both manual and automatic processes. Hand washing, the manual removal of stains from fabrics or from hard surfaces or the use in relation with an automatic process represent exemplary embodiments, wherein automatic processes, in particular for cleaning fabrics, are preferred due to their more precise controllability in regard to the added amounts and contact time, for example. Preferably, the above listed concentration ranges are correspondingly valid for these uses.

Fabric cleaning preferably occurs at temperatures of 20-95° C., preferably at temperatures of 20-60° C., in particular at temperatures of 20-40° C. Cleaning further occurs preferably at a pH of 5 to 12, in particular from 8 to 11.

Fabric cleaning processes are generally characterized as applying cleaning-active substances to material to be cleaned in a plurality of process steps and, after a contact time, are washed away, or that the material to be cleaned is treated in any other way with a washing agent or a solution of this agent. The same applies to methods for cleaning all materials other than fabrics, commonly referred to as “hard surfaces”. All conceivable washing or cleaning processes can be enriched in at least one of the process steps by an inventive bleach catalyst, and then represent embodiments of the present invention.

A preferred process includes providing the perhydrolase and C₂₋₆ alkylene glycol diacetate according to one of the above listed formulations for inventive agents, preferably washing or cleaning agents.

Another aspect of the present invention is a product comprising an inventive composition or inventive washing or cleaning agent, in particular an inventive cleaner for hard surfaces and a spray dispenser. In this regard, the product can be either a single chamber or a multi-chamber container, in particular a two-chamber container. The preferred spray dispenser is a manually operated spray dispenser such as aerosol spray dispensers (pressurized gas containers; also known inter alia as spray cans), self-generated pressure spray dispensers, pump spray dispensers and trigger spray dispensers. Particularly preferred are pump spray dispensers and trigger spray dispensers wherein the container is made of transparent polyethylene or polyethylene terephthalate. Spray dispensers are extensively described in International Patent Publication No. WO 96/04940 and the US patents cited therein concerning spray dispensers, all of which are referred to in this respect. Trigger spray dispensers and pump spray dispensers are advantageous in comparison with pressurized gas containers as no propellant need be employed. Using attachments suitable for particles (“nozzle-valves”) on the spray dispenser, enzymes in this embodiment can also be optionally added in the form of immobilized particles to the composition, and can thus be dosed as the cleaning foam.

As an example, inventively preferred liquid fabric-washing agents contain
0.3-0.5 wt. % xanthan gum;
0.2-0.4 wt. % anti-foam agent;
6-7 wt. % glycerin;
0.3-0.5 wt. % ethanol;
4-7 wt. % FAEOS;
24-28 wt. % non-ionic surfactants;
1 wt. % boric acid;
1-2 wt. % sodium citrate (dihydrate);
2-4 wt. % soda;
14-16 wt. % coconut fatty acids;
0.5 wt. % HEDP;
0.1 to 5 wt. %, preferably 0.2 to 3 wt. % perhydrolases, as well as optional additional enzyme(s), in particular, chosen from amylases, proteases and amadoriases;
0.01 to 5 wt. %, preferably 0.05 to 4 wt. % and in particular 0.1 to 3 wt. % C₂₋₆ alkylene glycol diacetates, in particular C₂₋₃ alkylene glycol diacetates;
optionally up to 0.4% PVP;
optionally up to 0.05% optical brightener; and
optionally up to 0.001% colorant.

As an example, inventively preferred powdered fabric-washing agents contain
10-15 wt. % LAS-Na;
2-5 wt. % FAEO;
0.5-2 wt. % FAS;
1-5 wt. % polyacrylates;
0.5-1 wt. % HEDP;
2.5-7 wt. % sodium silicate;
20-25 wt. % sodium carbonate;
2.5-7 wt. % sodium hydrogen carbonate;
10-20 wt. % sodium carbonate peroxo hydrate;
1-5 wt. % TAED;
0.25-1 wt. % CMC;
0.25-1 wt. % defoamer;
0.05-0.3 wt. % optical brightener;
0.1-0.5 wt. % fragrances;
0.1-5 wt. % perhydrolases;
0.01 to 5 wt. %, preferably 0.05 to 4 wt. %, in particular, 0.1 to 3 wt. % C₂₋₆ alkylene glycol diacetates, in particular C₂₋₃ alkylene glycol diacetates; and
sodium sulfate

Another example of a preferred fabric-washing agent comprises
15-18 wt. % linear alkylbenzene sulfonate;
8-12 wt. % non-ionic surfactant, in particular C₁₂₋₁₈ fatty alcohol ethoxylate (7 EO);
0.5-1.0 wt. % hydroxyethane diphosphonic acid;
2-4 wt. % sodium citrate;
6-10 wt. % sodium sulfate;
0.1-0.4 wt. % xanthan gum;
0.1 to 5 wt. %, preferably 0.2 to 3 wt. % perhydrolases, as well as optional additional enzyme(s), in particular, chosen from amylases, proteases and amadoriases;
0.01 to 5 wt. %, preferably 0.02 to 4 wt. % and particularly 0.05 to 3 wt. % C₂₋₆ alkylene glycol diacetates, in particular, C₂₋₃ alkylene glycol diacetates,
optionally up to 2 wt. % perfume; and
optionally up to 0.2 wt. % defoamer.

Inventively preferred automatic dishwasher detergents can contain, for example
5 to 70 wt. %, preferably 10 to 60 wt. % and especially 20 to 50 wt. % builder(s), other than washing and cleaning active polymers, preferably phosphates;
2 to 28 wt. %, preferably 4 to 20 wt. % and in particular 6 to 15 wt. % washing and cleaning active polymers;
0.5 to 10 wt. %, preferably 1 to 8 wt. % and especially 2 to 6 wt. % surfactant(s), preferably non-ionic and/or amphoteric surfactant(s), particularly preferably non-ionic surfactant(s);
0.5 to 8 wt. %, preferably 1 to 7 wt. % and especially 2 to 6 wt. % perhydrolases, as well as optional additional enzyme(s), in particular selected from amylases, proteases and amadoriases;
2 to 20 wt. %, preferably 4 to 15 wt. % and in particular 6 to 12 wt. % bleaching agent, preferably percarbonate;
optionally, 0.01 to 5 wt. %, preferably 0.02 to 4 wt. % and in particular 0.05 to 3 wt. % bleach catalysts; and
0.01 to 5 wt. %, preferably 0.05 to 4 wt. % and in particular 0.1 to 3 wt. % C₂₋₆ alkylene glycol diacetates, preferably C₂₋₃ alkylene glycol diacetates.

Particularly preferred automatic dishwasher agents comprise
5 to 70 wt. %, preferably 10 to 60 wt. % and in particular 20 to 50 wt. % phosphates;
2 to 28 wt. %, preferably 4 to 20 wt. % and in particular 6 to 15 wt. % washing and cleaning active polymers;
0.5 to 10 wt. %, preferably 1 to 8 wt. % and in particular, 2 to 6 wt. % non-ionic surfactant(s);
0.5 to 8 wt. %, preferably 1 to 7 wt. % and especially 2 to 6 wt. % perhydrolases, as well as optional additional enzyme(s), in particular, chosen from amylases, proteases and amadoriases;
2 to 20 wt. %, preferably 4 to 15 wt. % and in particular 6 to 12 wt. % percarbonate;
optionally 0.01 to 5 wt. %, preferably 0.02 to 4 wt. % and in particular 0.05 to 3 wt. % bleach catalysts; and
0.01 to 5 wt. %, preferably 0.05 to 4 wt. % and in particular 0.1 to 3 wt. % C₂₋₆ alkylene glycol diacetates, preferably C₂₋₃ alkylene glycol diacetates.

Automatic dishwasher detergents according to the invention can be produced in various ways. They can be in solid or liquid form, as well as in combined solid and liquid presentation forms. Powders, granulates, extrudates or compactates, especially tablets, are suitable solid presentation forms. Liquid presentation forms based on water and/or organic solvents can be thickened in the form of gels.

Agents according to the invention can be produced as one-phase or multi-phase products. Automatic dishwasher detergents with one, two, three or four phases are especially preferred. Automatic dishwasher detergents in the form of a prefabricated unit dose with two or more phases are particularly preferred.

Individual phases of a multi-phase agent can have the same or different aggregation states. Automatic dishwasher detergents that have at least two different solid phases and/or at least two liquid phases and/or at least one solid and at least one liquid phase are particularly preferred.

Automatic dishwasher detergents according to the invention are preferably prefabricated in a unit dose form. These unit doses preferably contain the necessary quantity of washing or cleaning active substances for one cleaning cycle. Preferred unit doses weigh from 12 to 30 g (grams), preferably from 14 to 26 g, and especially from 16 to 22 g.

The volumes of the abovementioned unit doses and their three-dimensional shape are particularly preferably chosen such that the prefabricated units can be dosed by being placed in the dosing chamber of a dishwasher. Consequently, the volume of the unit dose is preferably from 10 to 35 ml, preferably from 12 to 30 ml and especially from 15 to 25 ml.

Automatic dishwasher detergents according to the invention, in particular, the prefabricated unit doses, preferably have a water-soluble coating.

### Cosmetic and Pharmaceutical Preparations

In a further preferred embodiment, compositions comprising perhydrolase and C₂₋₆ alkylene glycol diacetate include cosmetic or pharmaceutical preparations. Such pharmaceutical compositions can be employed both anaphylactically as well as prophylactically.

When manufacturing pharmaceutical preparations, active substances, optionally in combination with other active principals, can be incorporated into typical galenical preparations such as tablets, dragees (sugar-coated), capsules, powders, suspensions, drops, ampoules, juices or suppositories with one or more inert, conventional carriers and/or diluents (e.g., with gelatin, gum arabic, corn starch, milk sugar, raw sugar, sorbitol, microcrystalline cellulose, magnesium stearate, polyvinyl pyrrolidone, citric acid, tartaric acid, water, benzyl alcohol, polyalkylene glycol, water/ethanol, water/glycerin, water/sorbitol, water/polyethylene glycol, propylene glycol, titanium dioxide, a cellulose derivative such as carboxymethyl cellulose or fat-containing substances like hydrogenated fat, talcum or vegetal oils or their appropriate mixtures). Optionally, preservatives, stabilizers, wetting agents, emulsifiers or salts for modifying the osmotic pressure or buffers can be included. Interfacially active auxiliaries such as salts of gallic acid or animal or vegetal phospholipids, mixtures thereof, as well as liposomes or their constituents can also be used as the carrier.

In a preferred embodiment, the pharmaceutical or cosmetic preparations concern those topically applied on the skin and their adnexa and/or for application on the mucous membrane, particularly, orally. These preparations are designated herein as “skin treatment agents”.

Cosmetic or pharmaceutical preparations, particularly skin treatment agents, can be in the form of a lotion, a cream, an emulsion, a balm, a paste, an oil, a wax/fat compound, a gel, a powder, a spray or aerosol, a solution, particularly aqueous or alcoholic solution, or tincture, a moist dressing, an occlusal dressing, a plaster, a stick preparation, a hair treatment, hair washing or hair care product, particularly a hair shampoo, a hair lotion, a hair cure or a hair water, a bubble bath, a shower bath or a foot bath. Physiological carriers for skin treatment agents advantageously include one or, in any combination, a plurality of auxiliaries normally used in such preparations, such as fats, oils, greasing materials, waxes, silicones, emulsifiers, dispersants, pearlizers, alcohols, polyols, consistency agents, stabilizers, thickeners, film formers, swelling agents, hydrotropes or moisturizers and/or humectants, polymers, surfactants, plasticizers, defoamers, alkali- or acidifiers, water softeners, adsorbents, light stabilizers, electrolytes, sequestering agents, solubilizers, organic solvents, preservatives, germicides, particularly fungicides or bactericides, antioxidants, biogenic active substances, vitamins, protein hydrolyzates, mono-, oligo- and polysaccharides, enzyme inhibitors, particularly MMP1-inhibiting substances, deodorants or odor absorbers, antiperspirants, antidandruff agents, insect repellents, self-tanning lotions, α-hydroxy- and α-ketocarboxylic acids, fragrances, colorants and/or pigments.

The skin treatment agents are advantageously present for topical administration in the form of a liquid or solid oil-in-water emulsion, water-in-oil emulsion, multiple emulsion, micro-emulsion, PIT-emulsion or Pickering emulsion, hydrogel, an alcoholic gel, a lipogel, a mono or multiphase solution, a foam, a balm, a plaster, a suspension, a powder or a mixture with at least one polymer that is a suitable medicinal adhesive. The skin treatment agents can also be presented in an anhydrous state, such as in an oil or balsam. For this, the carrier can be vegetable or animal oil, mineral oil, synthetic oil or a mixture of such oils.

In a further preferred embodiment according to the invention, cosmetic and/or pharmaceutical preparations include those for oral application, wherein the target areas of the application are the mouth itself or the teeth. In a preferred embodiment, one of the previously described skin treatment agents is used, wherein the composition is in the form of a mouth cream, balm, tincture or suspension. The term, “pharmaceutical preparation for oral application” also includes, in addition to mouth and teeth care agents, prosthesis cleansing agents, particularly, cleansing tablets for dentures.

Because of their oxidative properties, compositions according to the invention can be used for cleaning and/or bleaching teeth. Consequently, a further subject matter of the present invention includes the use of at least one perhydrolase together with at least one C₂₋₆ alkylene glycol diacetate for manufacturing a pharmaceutical and/or cosmetic preparation, in particular for cleaning and/or brightening and/or bleaching teeth and/or for producing an improved bleaching effect on teeth, as well as use of cosmetic compositions according to the invention for cleaning and/or brightening and/or bleaching teeth and/or for producing an improved bleaching effect on teeth.

Inventive oral, dental and/or dental prostheses care compositions can be in the form of mouthwash, gels, liquid toothpaste, viscous toothpaste, denture cleaners or adhesive creams for prostheses. For this, the inventively used materials must be proposed in a suitable carrier.

Moreover, in the oral region, mouthwashes, toothpastes, tablets, particularly lozenges as well as sprays or aerosols are further preferred embodiments.

For partial dentures or dentures, the presentation is suitable both as denture cleaning tablets and also as mouth rinses or mouth wash, or as toothpaste.

In addition to the inventive active substance, the inventive toothpastes and tooth gels can include surfactants, cleaning compounds, aromas, sweeteners, and additional active substances known in the art. Water and binders advantageously serve as carriers. Furthermore, humectants, preservatives, consistency agents and/or color pigments, for example, can also be included.

The inventive mouth wash can be in the form of aqueous, in particular, alcohol-containing, aromaticized concentrates or as ready-for-use solutions. In addition to the inventive active substances, the mouth waters can include surfactants, aromas, colorants, fluorides, astringent substances, antibacterials and/or additional active substances.

Regarding the additional active ingredients that may be included in the oral treatment agents, they include, for example, a fluorine compound, an active substance against plaque bacteria, an active substance for tartar control, an active for remineralization, an aid against sensitive teeth or for protection of the gums. Moreover, additional active substance can include an additional active substance for fungal treatment, particularly, treatment of candidosis.

Examples of other typical additives for oral, dental and/or dental prostheses care agents include
pH adjustors and buffer substances such as sodium bicarbonate, sodium citrate, sodium benzoate, citric acid, phosphoric acid or acidic salts (e.g., NaH₂PO₄);
wound healing and anti-inflammatory substances such as allantoin, urea, panthenol, azulene or camomile extract;
further active materials against tartar such as organo phosphonates (e.g., hydroxyethane diphosphonates or azacycloheptane diphosphonate);
preservatives such as salts of sorbic acid, sodium benzoate, chlorhexidine digluconate, p-hydroxybenzoic acid or its esters; and
plaque-inhibitors such as hexachlorophene, chlorhexidine, hexetidine, triclosan, bromochlorophene, phenyl salicylate.

Particularly preferred embodiments for cosmetic or pharmaceutical preparations include an oral or dental treatment agent. Preferred embodiments of the oral and dental treatment agents are further discussed below.

### Oral and Dental Treatment Agents

In the context of the invention, oral and dental treatment agents refer to oral and/or dental care and/or oral and dental cleaning agents. They particularly refer to oral and dental powder, oral and dental pastes, liquid oral and dental creams, oral and dental gels, mouth water, chewing gums, dental plasters (strips), tooth picks, dental floss and bleaching pens. According to the invention, oral and dental care and oral and dental cleaning agents are also understood to mean dental prostheses care agents, in particular, denture cleaners and adhesive creams for prostheses.

In a preferred embodiment, agents according to the invention are more or less free-flowing or plastic toothpastes, as are used with toothbrushes for cleaning teeth. The pH of the oral and dental treatment agents is preferably from 4 to 9, particularly preferably from 5 to 7.

Oral and dental care and oral and dental cleaning agents according to the invention can further comprise additional ingredients of mouth cleaners, oral treatment care agents, dental cleaning agents and/or dental care agents. Examples of these additional ingredients and their preferred addition quantities can be found in, for example, International Patent Publication No. WO 2009/015951.

Enzymes according to the invention can be particularly advantageously utilized in liquid or semi-liquid teeth cleaning preparations, particularly in translucent or transparent formulations with high consumer acceptance. In a particularly preferred embodiment, the enzymes can be stably incorporated into a special matrix, wherein the cleaning power of the corresponding agent —that can also be formulated to a transparent mixture—is superior to conventional agents.

Accordingly, a further preferred subject matter of the present invention is an oral and dental care and oral and dental cleaning agent comprising, in addition to at least one of the abovementioned enzymes according to the invention, 15 to 35 wt. % water together with 35 to 55 wt. % of at least one polyhydric alcohol from the group of sorbitol and/or glycerin and/or 1,2-propylene glycol, each based on weight of the agent. Naturally in addition, further ingredients, as cited above, can also be included. In this embodiment, preferred oral and dental care and oral and dental cleaning agents comprise 20 to 34 wt. %, preferably 22.5 to 33 wt. %, particularly preferably 24 to 32 wt. % and in particular 25 to 31 wt. % water, based on total weight of the care or cleaning agent. In determining total water content of the agent, the water fractions of the aqueous solutions are included. For example, if sorbitol is used in the form of a 70 wt. % concentrate solution, then the sorbitol fraction is 0.7 times the added weight fraction, whereas the water fraction is increased by 0.3 times the added weight fraction. The same applies to aqueous solutions of colorants or aromas, etc.

In this preferred embodiment, oral and dental care and oral and dental cleaning agents according to the invention include at least one polyhydric alcohol from the group sorbitol, glycerin and 1,2-propylene glycol as an additional essential ingredient in an amount of 35 to 55 wt. %, based on total weight of the composition. Here, preferred oral and dental care and oral and dental cleaning agents according to the invention comprise 37.5 to 52.5 wt. %, preferably 39 to 51 wt. %, particularly preferably 40 to 50 wt. % and particularly 42 to 49 wt. % of at least one polyhydric alcohol from the group sorbitol and/or glycerin and/or 1,2-propylene glycol.

In a further preferred embodiment, the agent according to the invention is a denture cleaner or a denture adhesive. For preferred denture cleaners, particularly denture cleaning tablets and powders, besides those ingredients already mentioned for oral, dental and/or dental prostheses care, peroxy compounds such as peroxyborate, peroxymonosulfate or percarbonate are also suitable. They have the advantage that, in addition to their bleaching activity, they also act as deodorizers and/or as disinfectants. Such peroxy compounds are added in denture cleaners in a range of from 0.01 to 10 wt. %, particularly from 0.5 to 5 wt. %. The pH of the denture cleaner can be from pH 4 to pH 12, in particular from pH 5 to pH 11.

Further auxiliaries are included in the denture cleaning tablets, such as agents that are effervescent in nature (e.g., CO₂-releasing materials such as sodium hydrogen carbonate), fillers (e.g., sodium sulfate or dextrose), lubricants (e.g., magnesium stearate), flow regulators such as colloidal silicon dioxide and granulating agents, such as high molecular weight polyethylene glycols or polyvinyl pyrrolidone.

Denture adhesives can be offered as powders, creams, films or liquids and support the adhesion of the dentures. Natural and synthetic swelling agents are suitable as active principles. Besides alginates, vegetal gums such as gum arabicum, traganth and karayi gum as well as natural rubber are to be understood as natural swelling agents. In particular, alginates and synthetic swelling agents such as sodium carboxymethyl cellulose, high molecular weight ethylene oxide copolymers, salts of polyvinyl ether-maleic acid and polyacrylamides have proven to be particularly suitable.

In particular, hydrophobic foundations, especially hydrocarbons such as white Vaseline (DAB) or paraffin oil, are especially suitable as auxiliaries for pasty and liquid products. The following examples further exemplify the present invention without limiting it in any way.

### Examples

### Example 1

### Specific Activity of Glycol Diacetates

TABLE 1
Summary of the specific activities of perhydrolase substrates

| Name | Structure | Mol-equiv. |
|---|---|---|
| Ethylene glycol diacetate | CH₂COOCH₂CH₂OCOCH₃ | 1.00 |
| Propylene glycol diacetate | CH₃COOCH₂CH(CH₃)OCOCH₃ | 0.91 |
| 3,4-Diacetoxy-2-butene | CH₃COOCH₂CH═CHCH₂OCOCH₃ | 0.85 |
| Diacetin | (CH₃COOCH₂)₂CHOH | 0.83 |
| 2-(1-Methoxy)propyl acetate | CH₃COOCH(CH₃)CH₂OCH₃ | 0.55 |
| Monoacetin | CH₃COOCH₂(CH₂OH)₂ | 0.41 |
| Diethylene glycol monoethyl | CH₃CH₂OCH₂CH₂OCH₂CH₂OCOCH₃ | 0.41 |
| ether acetate | | |
| Diethylene glycol mono-n-butyl | CH₃COOCH₂CH₂OCH₂CH₂O(CH₂)₃CH₃ | 0.36 |
| ether acetate | | |

By setting the number of functional groups as a ratio of the molecular weight of each of the substances, it becomes clear that ethylene glycol diacetate and propylene glycol diacetate carry more functional groups per unit of weight than the other known perhydrolase substrates. Consequently, lower amounts of these substrates should added. In Table 1, the ratio of functional groups to molecular weight of the ethylene glycol diacetate was set to equal one.

### Example 2

### Washing Results with Ethylene Glycol Diacetate and Propylene Glycol Diacetate

### a) Tested Stains:

blueberry juice on cotton: product no. C15 from CFT B. V. Vlaardingen, Holland
tea on cotton: product no. 167, Eidgenössische Material- and Prüfanstalt (EMPA) Testmaterialien AG, St. Gallen, Switzerland

### b) Fabric-Washing Agent Used:

TABLE 2
Formulation of the fabric-washing agent

| Chemical name | wt. % pure substance |
|---|---|
| Xanthan Gum | 0.3-0.5 |
| Antifoaming agent | 0.2-0.4 |
| Glycerin | 6-7 |
| Ethanol | 0.3-0.5 |
| FAEOS | 4-7 |
| Non-ionic surfactant (FAEO, APG, i.a.) | 24-28 |
| Boric acid | 1 |
| Sodium citrate × 2H₂O | 1-2 |
| Caustic soda | 2-4 |
| Coconut fatty acid | 14-16 |
| HEDP | 0.5 |
| PVP | 0-0.4 |
| Optical brightener | 0-0.05 |
| Dye | 0-0.001 |
| Perfume | 0-2 |
| Enzymes (Protease, Amylase, Cellulase) | 0.1-5 |
| H₂O, demin. | Remainder |

### c) Test Material (1 ml) in 48 Well Plates:

| Volume | Solution |
|---|---|
| 420 μl | 161-966 mg fabric washing agent in 42 ml water or |
| | buffer |
| 30-530 μl | 1-100 U/ml perhydrolase |
| 1-150 mM | Substrate (final concentration in the alkaline solution) |
| Remainder | H₂O |
| | Stain Ø 1 cm |

### d) Incubation:

60 min., 40° C., ca. 600 rpm

After incubation, the stains were washed three times (3×), dried and fixed. Intensity was measured with a colorimeter Cm508d (Minolta). For this test, round pieces of the test fabric (diameter 10 mm) were incubated in 1 ml alkaline solution in a 24 well microtitration plate. Each test was carried out as a triple determination against a triply determined control. After washing, the degree of whiteness of the washed fabrics was measured in comparison with a whiteness standard (d/8, Ø 8 mm, SCl/SCE), which had been set at 100% (L-value determination). The measurement was made using a colorimeter (Minolta Cm508d) with a light setting of 10°/D65. The results were expressed as percent power, wherein the difference of the reflectance value of the base washing agent without enzymes but with substrate was normalized to 100% (with the comparison substrate methyl acetate).

### e) Results:

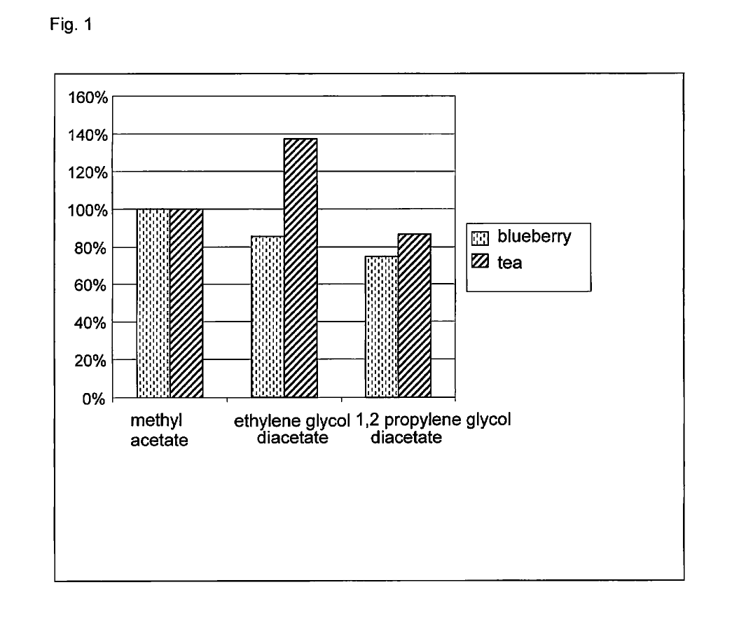

The washing results from the use of each 50 mM substrate (ethylene glycol diacetate and propylene glycol diacetate in comparison with methyl acetate) are shown in FIG. 1. It is clear that ethylene glycol diacetate (addition quantity 50 mM) delivers a significantly better washing power for tea stains than methyl acetate, and the other washing results are comparable. Ethylene glycol diacetate and propylene glycol diacetate, however, have a great advantage over methyl acetate in that they are not organic solvents. Moreover, in contrast to methyl acetate, they are highly water-soluble, odorless, non-toxic and non-volatile.

### Example 3

### Exemplary Formulations

### a) General Formulation for a Liquid Fabric-Washing Agent:

| Chemical name | wt. % pure substance |
|---|---|
| Xanthan Gum | 0.3-0.5 |
| Antifoaming agent | 0.2-0.4 |
| Glycerin | 6-7 |
| Ethanol | 0.3-0.5 |
| FAEOS | 4-7 |
| Non-ionic surfactant (FAEO, APG, i.a.) | 24-28 |
| Boric acid | 1 |
| Sodium citrate × 2H₂O | 1-2 |
| Caustic soda | 2-4 |
| Coconut fatty acid | 14-16 |
| HEDP | 0.5 |
| PVP | 0-0.4 |
| Optical brightener | 0-0.05 |
| Dye | 0-0.001 |
| Perfume | 0-2 |
| Enzymes (perhydrolase and optionally | 0.1-5 |
| protease, amylase and/or cellulase) | |
| Ethylene glycol diacetate | 0.1-5 |
| H₂O, demin. | Remainder |

In the formulation, FAEOS stands for sulfated fatty alcohol ethoxylate and FAEO for fatty alcohol ethoxylate, in particular for C₁₂₋₁₈ fatty alcohol ethoxylate (6-8 EO), APG for alkyl polyglycoside, HEDP for hydroxyethane-1,1-diphosphonic acid and PVP for polyvinyl pyrrol idone.

### b) General Formulation for a Powder Fabric-Washing Agent:

| Chemical name | wt. % pure substance |
|---|---|
| LAS-Na | 10-15 |
| FAEO | 2-5 |
| FAS | 0.5-2 |
| Polyacrylate | 1-5 |
| HEDP salt | 0.5-1 |
| Sodium silicate modulus 2.0 | 2.5-7 |
| Sodium carbonate | 20-25 |
| Sodium hydrogen carbonate | 2.5-7 |
| Sodium carbonate peroxohydrate | 10-20 |
| TAED | 1-5 |
| CMC | 0.25-1 |
| Defoamer | 0.25-1 |
| Optical brightener | 0.05-0.3 |
| Fragrance | 0.1-0.5 |
| Enzymes (perhydrolase and optionally | 0.1-5 |
| protease, amylase and/or cellulase) | |
| Ethylene glycol diacetate | 0.1-5 |
| Sodium sulphate | Remainder |

In the formulation, LAS stands for linear alkylbenzene sulfonate, FAEO for fatty alcohol ethoxylate, in particular for C₁₂₋₁₈ fatty alcohol ethoxylate (6-8 EO), FAS for fatty alcohol sulfate, HEDP for hydroxyethane-1,1-diphosphonic acid, TAED for N,N,N′,N′-tetraacetyl ethylenediamine and CMC for carboxymethyl cellulose.

### c) Exemplary Formulation for Washing and Cleaning Agents:

| Quantity | |
|---|---|
| [wt. % | |
| active | |
| principle] | Ingredient |
| 16.5 | Linear sodium alkylbenzene sulfonate (commercial product |
| | Maranil ®; Cognis, Dusseldorf) |
| 10 | Non-ionic surfactant (C₁₂₋₁₈ fatty alcohol ethoxylate (7 EO); |
| | commercial product Dehydol ® LT 7; Cognis) |
| 1 | Hydroxyethane-1,1-diphosphonic acid, Na4 salt (HEDP; |
| | Sequion ® 10 H 60; Polygon Chemie, Olten, Switzerland) |
| 3 | Sodium citrate |
| 8 | Sodium sulfate |
| 3 | Ethylene glycol diacetate |
| 3.5 | Enzyme granulate comprising perhydrolase |
| 0.25 | Xanthane gum; commercial product TGCS; Jungbunzlauer |
| | Xanthan, Pernhofen, Austria) |
| 1 | Perfume |
| 0.1 | Silicone defoamer (commercial product DC 2-3910; Wacker, |
| | Munich) |
| ad 100 | Water |

### 2) Exemplary Formulations for Dental Treatment Agents:

The following formulations are examples of dental treatment agents according to the invention. The quantities of the components are given in wt. %, each based on total composition.

| Composition | 1 | 2 | 3 | 4 | 5 |
|---|---|---|---|---|---|
| Sident 8 | 14.0 | — | — | — | — |
| Sorbosil AC 39 | — | 14.0 | — | — | 14.0 |
| Zeodent 113 | — | — | 14.0 | — | — |
| Zeodent 623 | — | — | — | 14.0 | — |
| Polishing clay P10 finest | 1.0 | 1.0 | 1.0 | 1.0 | 0.5 |
| Na₅P₃O₁₀ | 5.0 | 5.0 | 5.0 | 5.0 | 5.0 |
| (Na tripolyphosphate) | | | | | |
| Na₂PO₃F (Na | — | 0.8 | — | — | — |
| monofluorophosphate) | | | | | |
| NaF | 0.24 | — | 0.24 | 0.24 | 0.24 |
| Na saccharinate | 0.1 | 0.1 | 0.1 | 0.1 | 0.1 |
| Titanium dioxide | 0.5 | 0.5 | 0.5 | 0.5 | 0.5 |
| PHB methyl ester | 0.1 | 0.1 | 0.1 | 0.1 | 0.1 |
| Carboxymethyl cellulose | 1.25 | 1.25 | 1.25 | 1.25 | 1.25 |
| Sorbitol (70% conc.) | 32.0 | 32.0 | 32.0 | 32.0 | 32.0 |
| 1,2-Propylene glycol | 5.0 | 5.0 | 5.0 | 5.0 | 5.0 |
| Cremophor RH 60 | 1.0 | — | 1.0 | 1.0 | 1.0 |
| Arlatone 289 | — | 1.0 | — | — | — |
| Aroma oil | 0.8 | 0.8 | 0.8 | 0.8 | 0.8 |
| Perhydrolase | 0.3 | 0.5 | 0.5 | 0.7 | 0.3 |
| Ethylene glycol | 0.5 | 0.7 | 0.8 | — | — |
| diacetate | | | | | |
| Propylene glycol | — | — | — | 0.7 | 1.0 |
| diacetate | | | | | |
| Water | ad 100 | ad 100 | ad 100 | ad 100 | ad 100 |

| Composition | 6 | 7 | 8 | 9 |
|---|---|---|---|---|
| Sorbosil AC 39 | 14 | 14 | 14 | 14 |
| Polishing clay P10 finest | 1 | — | — | — |
| Precarb 100 | — | 2 | 2 | 2 |
| Na₅P₃O₁₀ | 10 | 10 | 5 | 0 |
| Na₂PO₃F | 0.8 | 0.8 | 0.8 | 0.8 |
| NaF | — | — | — | — |
| Na saccharinate | 0.1 | 0.1 | 0.1 | 0.1 |
| Titanium dioxide | 0.5 | 0.5 | 0.5 | 0.5 |
| PHB methyl ester | 0.1 | 0.1 | 0.1 | 0.1 |
| Carboxymethyl cellulose | 1.25 | 1.25 | 1.25 | 1.25 |
| Sorbitol (70% conc.) | 32.0 | 32.0 | 32.0 | 32.0 |
| 1,2-Propylene glycol | 5.0 | 5.0 | 5.0 | 5.0 |
| Arlatone 289 | 1.0 | 1.0 | 1.0 | 1.0 |
| Aroma oil | 0.8 | 0.8 | 0.8 | 0.8 |
| Perhydrolase | 0.2 | 0.5 | 0.6 | 0.8 |
| Ethylene glycol diacetate | 0.5 | 0.8 | — | — |
| Propylene glycol diacetate | — | — | 0.5 | 0.8 |
| Water | ad 100 | ad 100 | ad 100 | ad 100 |

| Composition | 10 | 11 | 12 | 13 |
|---|---|---|---|---|
| Sident ® 8 | 10.0 | 10.0 | 10.0 | 10.0 |
| Sident ® 22S | 7.0 | 7.0 | 7.0 | 7.0 |
| Sipernat ® 320DS | 0.8 | 0.8 | 0.8 | 0.8 |
| Glycerin solution comprising | 1.0 | 0.1 | 1.0 | 0.1 |
| 10 wt. % hydroxylapatite | | | | |
| Polywachs ® 1550 | 2.0 | 2.0 | 2.0 | 2.0 |
| Texapon ® K 1296 | 1.5 | 1.5 | 1.5 | 1.5 |
| Titanium dioxide | 1.0 | 1.0 | 1.0 | 1.0 |
| Cekol ® 500 T | 1.0 | 1.0 | 1.0 | 1.0 |
| Na fluoride | 0.33 | 0.33 | 0.33 | 0.33 |
| Na benzoate | 0.25 | 0.25 | 0.25 | 0.25 |
| Aroma | 1.0 | 1.0 | 1.0 | 1.0 |
| Tagat ® S | 0.2 | — | — | 0.2 |
| Na saccharinate | 0.15 | 0.15 | 0.15 | 0.15 |
| Trisodium phosphate | 0.10 | 0.10 | 0.10 | 0.10 |
| Sorbitol | 31.0 | 31.0 | 31.0 | 31.0 |
| (70% conc. in water) | | | | |
| Perhydrolase | 0.2 | 0.5 | 0.6 | 0.8 |
| Ethylene glycol diacetate | 0.5 | 0.8 | — | — |
| Propylene glycol diacetate | — | — | 0.5 | 0.8 |
| Water | ad 100 | ad 100 | ad 100 | ad 100 |

2-in-1 Formulations:

| Composition | 14 | 15 | 16 | 17 | 18 |
|---|---|---|---|---|---|
| Sorbitol (70% DAB) | 60 | 62.5 | 65 | 67.5 | 70.0 |
| Ethanol (96%) | — | — | — | — | 2.0 |
| Precipitated silica: Sident 8 | 10.0 | 10.0 | 10.0 | 10.0 | 12.0 |
| Precipitated silica: Sident 22S | 8.5 | 8.5 | 8.5 | 8.5 | — |
| Polishing clay P10 finest | — | 1.0 | — | — | — |
| Disodium phosphate, anhydrous | 0.1 | 0.1 | 0.1 | 0.1 | 0.1 |
| Trisodium phosphate, anhydrous | 0.2 | 0.2 | 0.2 | 0.2 | — |
| Na monofluorophosphate Na₂PO₃F | — | — | 1.0 | 1.0 | 1.0 |
| Sodium fluoride | 0.32 | 0.32 | — | — | — |
| Polyethylene glycol (mwt: 1500) | 1.5 | 1.5 | 1.5 | 1.5 | 1.5 |
| Titanium dioxide | 0.5 | 0.5 | 0.5 | 0.5 | — |
| Na lauryl sulfate | 1.5 | 1.5 | 1.5 | 1.5 | 1.5 |
| Saccharine Na | 0.1 | 0.1 | 0.1 | 0.1 | 0.1 |
| Xanthan | 0.4 | 0.4 | 0.4 | 0.4 | 0.4 |
| Aroma | 1.2 | 1.2 | 1.2 | 1.2 | 1.2 |
| Perhydrolase | 0.3 | 0.5 | 0.5 | 0.7 | 0.3 |
| Ethylene glycol diacetate | 0.5 | 0.7 | 0.8 | — | — |
| Propylene glycol diacetate | — | — | — | 0.7 | 1.0 |
| Water | ad 100 | ad 100 | ad 100 | ad 100 | ad 100 |

| Composition | 19 | 20 | 21 | 22 |
|---|---|---|---|---|
| Sident ® 8 | 14.0 | 14.0 | 14.0 | 14.0 |
| Sorbosil ® AC 33 | 1.0 | 1.0 | — | — |
| Polishing clay P10 finest | — | — | 1.0 | 1.0 |
| Glycerin (86% conc. DAB) | 18.0 | 18.0 | 18.0 | 18.0 |
| Sorbitol (70% conc.) | 14.0 | 14.0 | 14.0 | 14.0 |
| 1,2-Propylene glycol | 5.0 | 5.0 | 5.0 | 5.0 |
| AHP 100 | 0.5 | 0.5 | 0.5 | 0.5 |
| NaF | 0.32 | 0.32 | 0.32 | 0.32 |
| Saccharine Na | 0.2 | 0.2 | 0.2 | 0.2 |
| Na₂HPO₄ | 1.5 | 1.5 | 1.5 | 1.5 |
| Na₃PO₄ | 0.5 | 0.5 | 0.5 | 0.5 |
| TiO₂ | — | — | 0.5 | 0.5 |
| Timiron ® diamone Cluster MP 149 | 0.2 | 0.2 | — | — |
| Brilliant Blue 1% conc. (Acid Blue 9) | 0.22 | 0.22 | — | — |
| PHB methyl ester | 0.1 | 0.1 | 0.1 | 0.1 |
| Keltrol ® F | 1.25 | 1.25 | 1.25 | 1.25 |
| Texapon ® K1296 | 1.5 | 1.5 | 1.5 | 1.5 |
| Tego Betain ® BL 215 | 0.6 | 0.6 | 0.6 | 0.6 |
| Triclosan | 0.3 | — | 0.3 | — |
| Jojoba oil | 1.0 | 0.5 | 1.0 | 0.5 |
| Aroma oil | 1.15 | 1.15 | 1.0 | 1.0 |
| NaOH | 0.158 | 0.158 | 0.158 | 0.158 |
| Perhydrolase | 0.3 | 0.5 | 0.5 | 0.7 |
| Ethylene glycol diacetate | 0.5 | 0.7 | 0.8 | — |
| Propylene glycol diacetate | — | — | — | 0.7 |
| Water | ad 100 | ad 100 | ad 100 | ad 100 |

| Composition | 23 | 24 | 25 | 26 | 27 | 28 | 29 | 30 |
|---|---|---|---|---|---|---|---|---|
| Sident 8 | 12.0 | 12.0 | 12.0 | 12.0 | 12.0 | 12.0 | 12.0 | 12.0 |
| Sorbitol (70% conc.) | 30.0 | 30.0 | 30.0 | 30.0 | 30.0 | 30.0 | 30.0 | 30.0 |
| Glycerin 86% conc. | 33.0 | 33.0 | 33.0 | 33.0 | 33.0 | 33.0 | 33.0 | 33.0 |
| Lipoxol 1550 | 1.0 | 1.0 | 1.0 | 1.0 | 1.0 | 1.0 | 1.0 | 1.0 |
| Na saccharinate | 0.2 | 0.2 | 0.2 | 0.2 | 0.2 | 0.2 | 0.2 | 0.2 |
| Na benzoate | 0.5 | 0.5 | 0.5 | 0.5 | 0.5 | 0.5 | 0.5 | 0.5 |
| Na₂HPO₄ | 0.5 | 0.5 | 0.5 | 0.5 | 0.5 | 0.5 | 0.5 | 0.5 |
| Na fluoride | 0.32 | 0.32 | 0.32 | 0.32 | 0.32 | 0.32 | 0.32 | 0.32 |
| MgSO₄ 7H₂O | 0.21 | 0.21 | 0.21 | 0.21 | 0.21 | 0.21 | 0.21 | 0.21 |
| ZnSO₄ 7H₂O | 0.1 | 0.1 | 0.1 | 0.1 | 0.1 | 0.1 | 0.1 | 0.1 |
| MnSO₄ 7H₂O | 0.01 | 0.01 | 0.01 | 0.01 | 0.01 | 0.01 | 0.01 | 0.01 |
| Triclosan | 0.2 | 0.2 | 0.2 | 0;2 | 0;2 | 0;2 | 0;2 | 0;2 |
| Xanthan gum | 0.55 | 0.55 | 0.55 | 0.55 | 0.55 | 0.55 | 0.55 | 0.55 |
| Perfume | 1.0 | 1.0 | 1.0 | 1.0 | 1.0 | 1.0 | 1.0 | 1.0 |
| Texapon ® K1296 | 1.5 | 2.5 | 1.5 | 1.5 | 1.5 | 1.5 | 1.5 | 1.5 |
| Tagat ® S | 0.5 | 0.5 | 0.5 | 1.5 | 0.5 | 0.5 | 0.5 | 0.5 |
| Tego ® Betain BL215 | 0.6 | 0.6 | 3.8 | 0.6 | 0.6 | 0.6 | 0.6 | 0.6 |
| Gluadin ® W40 | — | — | — | — | 1.0 | — | — | — |
| Gluadin ® Almond | — | — | — | — | — | 1.0 | — | — |
| Gluadin ® R | — | — | — | — | — | — | 1.0 | — |
| Gluadin ® WK | — | — | — | — | — | — | — | 1.0 |
| Ethanol | 2.0 | 2.0 | 2.0 | 2.0 | 2.0 | 2.0 | 2.0 | 2.0 |
| Perhydrolase | 0.3 | 0.5 | 0.7 | 0.7 | 0.3 | 0.5 | 0.5 | 0.7 |
| Ethylene glycol | 0.5 | 0.7 | 0.9 | — | 0.5 | 0.7 | 0.9 | — |
| diacetate | | | | | | | | |
| Propylene glycol | — | — | — | 0.7 | — | — | — | 0.7 |
| diacetate | | | | | | | | |
| Water | ad 100 | ad. 100 | ad. 100 | ad. 100 | ad 100 | ad 100 | ad 100 | ad 100 |

The following commercial products were used:

| | |
|---|---|
| Plantacare ® 1200 | C₁₂-C₁₆ alkyl glycoside (Cognis) |
| Sident ® 8 | Synth. amorph. silica (Degussa) |
| Sident ® 22 S | Hydrogel silica (Degussa) |
| Polywachs ® 1550 | Polyethylene glycol 1550 (RWE/DEA) |
| Texapon ® K 1296 | Sodium lauryl sulfate (Cognis) |
| Cekol ® 500 T | Sodium carboxymethyl cellulose |
| | (Noviant) |
| Tagat ® S | Polyoxyethylene-(20)-glyceryl |
| | monostearate (Goldschmidt) |
| Sorbosil ® AC 39 | Silica abrasive (Crosfield) |
| Zeodent ® 113 | Hydrated Silica (Huber) |
| Zeodent ® 623 | Hydrated Silica (Huber) |
| Cremophor ® RH 60 | Polyoxyl 60 hydrogenated Castor oil |
| | (BASF) |
| Arlatone ® 289 | PEG-54 hydrogenated Castor oil |
| | (Uniqema) |
| Precarb 100 | Calcium carbonate |
| Timiron ® Diamond Cluster MP | Titanium dioxide (Merck) |
| 149 | |
| Keltrol ® F | Xanthan Gum (Keltrol) |
| Tego Betain ® BL 215 | Cocamidopropyl betaine (Goldschmidt) |
| Lipoxol ® 1550 | Polyethylene glycol 1550 (Sasol) |
| Tagat ® S | PEG-20 glyceryl stearate (Degussa) |
| Gluadin ® W40 | Hydrolyzed Wheat Protein (Cognis) |
| Gluadin ® Almond | Hydrolyzed Sweet Almond Protein |
| | (Cognis) |
| Gluadin ® R | Hydrolyzed Rice Protein (Cognis) |
| Gluadin ® WK | Sodium cocoyl Hydrolyzed Wheat |
| | Protein (Cognis) |

We claim:

1. Composition comprising at least one perhydrolase and at least one C₂₋₆ alkylene glycol diacetate.

2. Composition according to claim 1 wherein the C₂₋₆ alkylene glycol diacetate is ethylene glycol diacetate or propylene glycol diacetate or mixtures thereof.

3. Composition according to claim 1 further comprising hydrogen peroxide and/or a bleaching agent.

4. Composition according to claim 1 further comprising at least one hydrogen peroxide-producing oxidoreductase.

5. Composition according to claim 1 wherein said composition is a washing or cleaning agent.

6. Composition according to claim 5 wherein said composition is a surfactant-containing liquid or powder fabric-washing agent, a surfactant-containing automatic or manual dishwasher detergent or a surfactant-containing household cleaner.

7. Composition according to claim 1 wherein said composition is a solid, gelled, pasty or liquid composition.

8. Composition according to claim 5 further comprising:
5 to 70 wt. % builder(s), excluding washing and cleaning active polymers;
2 to 28 wt. % washing and cleaning active polymers;
0.5 to 10 wt. % surfactant(s), preferably non-ionic and/or amphoteric surfactant(s);
0.5 to 8 wt. % perhydrolases;
optional additional enzyme(s) chosen from amylases, proteases and amadoriases;
2 to 20 wt. % bleaching agent;
optionally 0.01 to 5 wt. % bleach catalysts; and
0.01 to 5 wt. % C₂₋₆ alkylene glycol diacetates.

9. Composition according to claim 5 further comprising:
10-15 wt. % LAS-Na;
2- 5 wt. % FAEO;
0.5-2 wt. % FAS;
1-5 wt. % polyacrylates;
0.5-1 wt. % HEDP;
2.5-7 wt. % sodium silicate;
20-25 wt. % sodium carbonate;
2.5-7 wt. % sodium hydrogen carbonate;
10-20 wt. % sodium carbonate peroxohydrate;
1-5 wt. % TAED;
0.25-1 wt. % CMC;
0.25-1 wt. % defoamer;
0.05-0.3 wt. % optical brightener;
0.1-0.5 wt. % fragrance;
0.01-5 wt. %. C₂₋₆ alkylene glycol diacetates;
0.1-5 wt. % perhydrolases; and
sodium sulfate.

10. Composition according to claim 5 further comprising:
0.3-0.5 wt. % Xanthane gum,
0.2-0.4 wt. % anti-foam agent,
6-7 wt. % glycerin,
0.3-0.5 wt. % ethanol,
4-7 wt. % FAEOS,
24-28 wt. % non-ionic surfactants
1 wt. % boric acid,
1-2 wt. % sodium citrate (dihydrate),
2- 4 wt. % soda,
14-16 wt. % coconut fatty acids,
0.5 wt. % HEDP,
0.1-5 wt. % perhydrolases, and
0.01-5 wt. %. C₂₋₆ alkylene glycol diacetates.

11. Composition according to claim 5 further comprising:
15-18 wt. % linear alkylbenzene sulfonate,
8-12 wt. % non-ionic surfactant, in particular C₁₂₋₁₈ fatty alcohol ethoxylate (7 EO),
0.5-1.0 wt. % hydroxyethane diphosphonic acid,
2-4 wt. % sodium citrate,
6-10 wt. % sodium sulfate,
0.1-0.4 wt. % Xanthane gum,
0.1 to 5 wt. % perhydrolases, and
0.01 to 5 wt. % C₂₋₆ alkylene glycol diacetates.

12. Composition according to claim 1 wherein said composition is a cosmetic or pharmaceutical preparation.
